(19) United States
(12) Patent Application Publication
(10) Pub. No.: US 2022/0125342 A1
(43) Pub. Date: Apr. 28, 2022

(54) SYSTEM AND METHOD FOR DIAGNOSING SUDDEN SENSORINEURAL HEARING LOSS

(71) Applicant: Taipei Veterans General Hospital, Taipei City (TW)

(72) Inventors: Wen-Huei LIAO, Taipei City (TW); Yen-Fu CHENG, Taipei City (TW); Yuan-Chia CHU, Taipei City (TW)

(73) Assignee: Taipei Veterans General Hospital, Taipei City (TW)

(21) Appl. No.: 17/512,436

(22) Filed: Oct. 27, 2021

Related U.S. Application Data
(60) Provisional application No. 63/106,050, filed on Oct. 27, 2020.

(51) Int. Cl. A61B 5/12; A61B 5/00

(52) U.S. Cl. CPC A61B 5/121; A61B 5/6898

(57) ABSTRACT

The present invention provides a computer-implanted method and system for diagnosing sudden sensorineural hearing loss (SSNHL) of an ear in a subject; comprising on one or more processors of the computer the steps of: testing the hearing of two ears of the subject, and calculating the difference between them; and comparing the difference with a threshold to diagnose the subject as SSNHL or not.

### CROSS REFERENCE TO RELATED APPLICATIONS

This application claims the benefit of and priority to U.S. Provisional Application No. 63/106,050, filed Oct. 27, 2020.

### FIELD OF THE INVENTION

The present invention provides a system and method for diagnosing sudden sensorineural hearing loss (SSNHL) in a subject.

### BACKGROUND OF THE INVENTION

Sudden sensorineural hearing loss (SSNHL) is an otologic emergency that warrants urgent clinical visits and timely management. SSNHL is commonly defined as a sensorineural hearing loss of 30 or more decibels (dB) over at least three consecutive audiometric frequencies occurring within a 72-hour period [1]; it affects approximately 5-27 per 100,000 people annually, and its incidence is gradually increasing over time [1-4]. Although SSNHL can occur at any age, the peak incidence occurs among adults aged 45 to 64 years, which is the general age range of working individuals [5]. The typical manifestations of SSNHL include immediate or rapidly progressive hearing loss and, sometimes, hearing loss upon awakening [1]. However, many patients with SSNHL often initially experience only nonspecific symptoms, such as aural fullness or a sensation of a blocked ear, and fail to recognize a loss of hearing, which results in delayed evaluations and treatment [1]. Compounded with the effects of aging and associated symptoms such as dizziness and tinnitus, SSNHL significantly impacts individuals' general health and quality of life and causes a considerable health care burden [1,6]. Previous studies have identified possible prognostic factors for hearing recovery following SSNHL, including age, the severity of hearing loss, the duration of hearing loss, and delay in treatment [5,7,8]. As a potentially modifiable variable, shortening the time between the onset of hearing loss and adequate intervention is a crucial step in improving posttreatment hearing outcomes and minimizing other negative health consequences associated with hearing loss [9-12].

Currently, pure-tone audiometry remains the gold standard for evaluations of SSNHL since it not only reflects the severity of hearing loss but also provides a baseline hearing status for the assessment of recovery [5,8]. Conventional pure-tone audiometry usually requires a standard soundproof booth and a calibrated audiometer, is performed by a qualified audiologist, and takes approximately 10-20 minutes per patient to perform. Considering the strict requirements regarding equipment and hearing care professionals, the accessibility of timely hearing evaluations using conventional pure-tone audiometry can be limited, especially in primary care settings [13,14]. To address these challenges and optimize the utilization of hearing health care, the traditional model of hearing screening and health service delivery should be supplemented with more efficient and attainable approaches. For hearing care, a hybrid hearing clinic with both internet-based and in-person services has been implemented in prior research and revealed high patient satisfaction [15]. In terms of hearing screening, innovative telemedicine tools such as computer-assisted hearing tests [16-19] and mobile phone-based devices [20-23] have been introduced and investigated.

The Hearing Scale Test (HST) is a novel hearing screening tool derived from consecutive hearing screening procedures and is used to estimate the current hearing status of each ear; it is based on the concepts of the Landolt C vision-test chart [24,25]. With stratified hearing scales that represent various sound levels and four of the main frequencies in speech perception, 0.5, 1, 2, and 4 kHz, the HST not only precisely reflects an individual's hearing status but also has a computer-based design that enables outcome monitoring and patient surveillance [24]. The HST has demonstrated satisfactory feasibility and accuracy for hearing screening programs in pediatric populations in prior studies [24,25]. A recent study that integrated the HST into a smartphone-based application (Ear Scale) reported remarkable validity for hearing screening among school-aged children [26].

However, in clinical settings such as primary care practices and urgent care facilities, conventional pure-tone audiometry is unavailable.

### BRIEF SUMMARY OF THE INVENTION

Accordingly, the present invention provides a computer-implanted method for diagnosing sudden sensorineural hearing loss (SSNHL) of an ear in a subject; comprising on one or more processors of the computer the steps of:
(a) measuring environmental background noise;
(b) providing a test tone at a high frequency using a plurality of magnitude levels for testing the hearing of a first ear of the subject, wherein the test tone is a pure tone;
(c) if the subject passes the hearing test in the step (b), providing a plurality of test tones using a plurality of magnitude levels respectively for testing the hearing of the first ear of the subject to record the results as a first hearing scale;
(d) providing a plurality of test tones using a plurality of magnitude levels for testing the hearing of a second ear of the subject to record the results as a second hearing scale, wherein the test tone is a pure tone;
(e) calculating the difference between the first hearing scale and the second hearing scale; and
(f) comparing the difference with a threshold to diagnose the subject as SSNHL or not; wherein the threshold is defined as the difference being five magnitude levels.

In an embodiment of the invention, the computer is integrated in a mobile device, such as a smart phone or a pad.

In an embodiment of the invention, the test tone may be a pure tone at a frequency at 8000 Hz, 4000 Hz, 2000 Hz, 1000 Hz or 500 Hz, wherein the pure tone not less than 2000 Hz is defined as a tone at a high frequency.

In an embodiment of the invention, the first magnitude level is 20 or 25 decibel hearing level (dB HL) for each test tone.

In an embodiment, the difference of two test magnitude levels is 5 dB HL.

In an embodiment, the threshold is the difference between the first hearing scale and the second hearing scale (i.e., the difference between the hearing scales of the two ears of the subject) not less than 5 magnitude levels, i.e., 25 dB HL. In an example, the difference can be the difference between the average of the first ear's magnitude scales and that of the second ear's magnitude scales in more than one tests.

On the other hand, the present invention provides a computer aid system configured to diagnose SSNHL, the computer aid system comprising of:

a memory;

a microphone;

a headphone; and

one or more processors for performing the steps set forth in the method for diagnosing SSNHL according to the invention.

In a preferable embodiment, the one or more processors are implanted in a computer such as a desk computer or a notebook computer, or a mobile device such as a smartphone or a pad; preferably a smartphone.

In the present invention, the correlation between hearing outcomes measured by conventional pure-tone audiometry and those measured by the proposed smartphone-based Ear Scale app is determined, and the diagnostic validity of the hearing scale differences between the two ears, as obtained by the Ear Scale app, for SSNHL is determined. Then, a cohort of 88 participants with possible SSNHL who were referred to an otolaryngology clinic or emergency department at a tertiary medical center in Taipei, Taiwan was conducted between January 2018 and June 2019. All participants underwent hearing assessments with conventional pure-tone audiometry and the proposed smartphone-based Ear Scale app consecutively. The gold standard for diagnosing SSNHL was defined as the pure-tone average (PTA) difference between the two ears being equal to or greater than 25 dB HL. In one example of the invention, the pure-tone average (PTA) difference between the two ears being equal to 35 dB HL.

It was found in the present invention, the hearing results measured by the Ear Scale app were presented as 20 stratified hearing scales. The hearing scale difference between the two ears was estimated to detect SSNHL.

In the clinical study of the present invention composed of 88 adults with a mean age of 46 years and 50% females, the PTA measured by conventional pure-tone audiometry was strongly correlated with the hearing scale assessed by the Ear Scale app, with a Pearson's correlation coefficient of 0.88 (95% CI=0.82-0.92). The sensitivity of the 5-hearing scale difference (25 dB HL difference) between the impaired ear and the contralateral ear in diagnosing SSNHL was 95.5% (95% CI=87.5%-99.1%), with a specificity of 66.7% (95% CI=43.0%-85.4%).

It is concluded in the present invention that the smartphone-based Ear Scale app can be useful in the evaluation of SSNHL in clinical settings.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing will be provided by the USPTO upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the screenshots of the ear scale app instructions for the subjects and the hearing test procedures.

FIG. 2 shows the scatter plots demonstrating (a) the correlation between the pure-tone average (PTA) obtained by pure-tone audiometry (y-axis) and the Hearing Scale measured by the Ear Scale app (x-axis); (b) the correlation between the PTA differences and Hearing Scale differences between the impaired and contralateral ears.

FIG. 3 provides the box plot of the pure-tone average (PTA) difference (y-axis) in relation to the hearing scale difference (x-axis) between the impaired and contralateral ears; wherein the green line depicts the best-fitted mean PTA difference in relation to the Hearing Scale difference for the linear regression, and the green area represents the 95% confidence interval of the model (p<0.05, significant differences were found between each Hearing Scale difference group). The dashed line represents PTA differences of 30 dB (i.e. diagnostic gold standard for detecting SSNHL in the present invention).

FIG. 4 shows the hearing screening procedures used in the present invention.

### DETAILED DESCRIPTION OF THE INVENTION

The above summary of the present invention will be further described with reference to the embodiments of the following examples. However, it should not be understood that the content of the present invention is only limited to the following embodiments, and all the inventions based on the above-mentioned contents of the present invention belong to the scope of the present invention.

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

As used herein, the term “computer” refers to a device comprising one or more processors for performing the method of the invention, which can be, but not limited to, a computer such as a desk computer or a notebook computer, or a mobile device such as a smartphone or a pad; preferably a smartphone.

In the present invention, the first aim is to investigate the validity of a smartphone-based hearing screening application integrated with the novel HST for the assessment of SSNHL. This study confirmed that there is a strong correlation in hearing results between conventional pure-tone audiometry and the proposed smartphone-based Ear Scale app in a cohort of patients with possible SSNHL. The sensitivity of the hearing scale difference between the two ears measured by the Ear Scale app (95.5% for 5-hearing scale difference [25 dB HL] with a specificity of 66.7%, 92.5% for 6-hearing scale difference [30 dB HL] with a specificity of 85.7%, and 91.0% for 7-hearing scale difference [35 dB HL] with a specificity of 90.5%) in diagnosing SSNHL was high, suggesting that the smartphone-based approach can assist in the evaluation of SSNHL, particularly in clinical settings where conventional pure-tone audiometry is not available.

On the other hand, the present invention provides In this study, we developed an iOS-based smartphone hearing test app Ear Scale and evaluated its performance and feasibility as a hearing screening program for school-age children. We investigated the accuracy of the hearing tests conducted on mobile devices calibrated by RETSPLs for Apple EarPod. We compared the performance of the smartphone-based automated hearing screening with that of audiologist-assisted pure-tone audiometry (PTA) performed in a sound-treated booth. Different screening protocols, including those suggested by the AAP and ASHA, were also compared with the built-in HST protocol of the Ear Scale app.

### Example 1

Methods

Study Design and Population

This cross-sectional study was conducted at a tertiary medical center in Taipei, Taiwan, from January 2018 to June 2019. The sample size needed to reach a power of 0.8 was 82. We recruited 88 adults with possible SSNHL who visited either an otolaryngology outpatient clinic or emergency department. The study was approved by the Institutional Review Board of the Taipei Veteran General Hospital (2016-12-004BC). The investigators explained the research objectives and process, and written informed consent was obtained from all the patients enrolled. Instructions regarding the screening procedures and operations were provided by the trained examiners prior to each hearing screening test.

Hearing Measurements

Conventional Pure-Tone Audiometric Assessments

Pure-tone audiometry was administered by certified audiologists in the outpatient department. Otoscopy was performed to examine the clearness of the ear canal. Audiometric examinations were performed with a GSI 61 two-channel audiometer in a soundproof booth. Standard clinical methods (modified Hughson-Westlake Methods) were used to obtain pure-tone air conduction thresholds. To assess the reliability of the threshold measures, 1000 Hz was tested twice in each ear; participants with a >10 decibel hearing level (dB HL) change between measures were considered unreliable. The pure-tone average (PTA) was calculated using air conduction thresholds at 0.5, 1, 2, and 4 kHz in each ear. Each individual's pretreatment hearing status measured by conventional pure-tone audiometry was categorized into the following 5 grades according to the modified Siegel criteria for SSNHL [27]: Grade 1 (PTA≤25 dB HL), Grade 2 (PTA 26-45 dB HL), Grade 3 (PTA 46-75 dB HL), Grade 4 (PTA 76-90 dB HL), and Grade 5 (PTA>90 dB HL).

Smartphone-Based Hearing Screening App

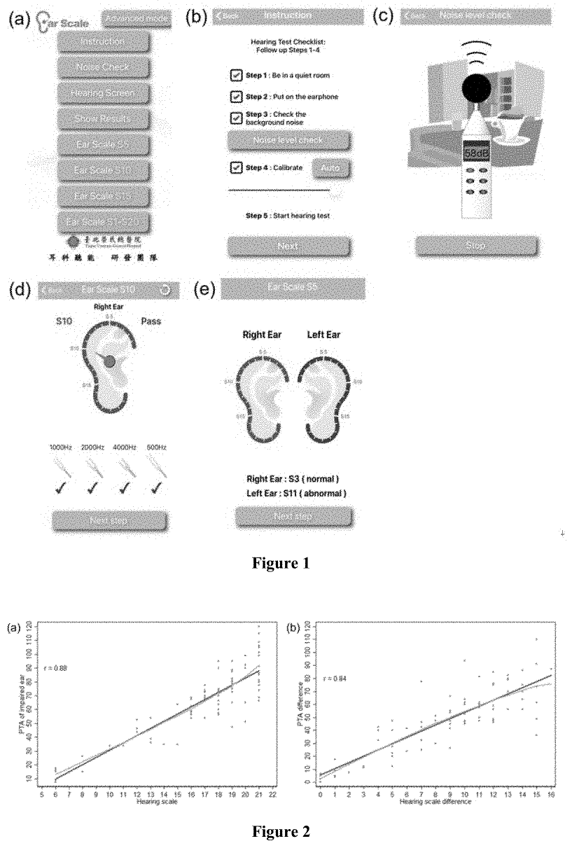

The mobile devices used in this study were the iPhone 7 or iPhone 7 plus, with iOS software version 13.3.2. The iOS-based automated Ear Scale app (version 2.0) was integrated with the HST and used to measure the hearing statuses of both ears in the enrolled participants (FIG. 1a). The items included in the hearing test checklist were assessed by the examiners (FIG. 1b). The patients were taught how to wear the headphones and click the response button when they heard the test tones. The headphones used throughout the examination were calibrated for Apple EarPods. The detailed calibration procedures are described in the next section. After the participants put on the headphones correctly, the background noise level was assessed immediately using the built-in function in the Ear Scale app to ensure that the ambient noise was less than 50 A-weighted decibels (dBA) (FIG. 1c). Lastly, the mobile device and headphones were calibrated and standardized before the HST was started (FIG. 1b). The HST incorporated in the Ear Scale app was a novel hearing screening tool developed on the basis of consecutive hearing screening procedures to estimate the current hearing status of each ear [24,25]. The HST measured individuals' hearing status with respect to stratified hearing scales that represented sound intensity and 4 test frequencies (0.5 kHz, 1 kHz, 2 kHz, and 4 kHz). The adjacent scales differed from one another by 5 dB HL (Table 1). The test tones lasted for 1.5 seconds, and the silent intervals lasted for 2 to 3 seconds [26,28]. The Ear Scale app started with Hearing Scale 5 (S₅), which corresponded to 25 dB HL. The four test tones were automatically presented to patients in an order of 1 kHz, 2 kHz, 4 kHz, and 0.5 kHz. The stimulus level of the pure tones descended to the next adjacent Hearing Scale only if the patient responded correctly to all the tones (FIG. 1d). The minimum audible Hearing Scale indicated the lowest pure-tone stimulus level at which the participant responded correctly to all four test tones, was shown at the end of each examination and was saved to the devices (FIG. 1e). The Hearing Scale difference between the impaired ear and the contralateral ear was determined and used for identifying patients with SSNHL (FIG. 1e).

TABLE 1
Sound volume in decibels for each stimulation level examined
in the Hearing Scale Test (HST) and for different frequencies

| | Hearing Scale Test (HST) | | | | | | | | | | | | | | | | | | | |
|---|---|---|---|---|---|---|---|---|---|---|---|---|---|---|---|---|---|---|---|---|
| | Pretreatment hearing grade [symbol] | | | | | | | | | | | | | | | | | | | |
| | Grade 1 | | | | | Grade 2 | | | | | Grade 3 | | | | | Grade 4 | | | Grade 5 | |
| | (PTA ≤ 25 dB HL) | | | | | (PTA 26-45 dB HL) | | | | | (PTA 46-75 dB HL) | | | | | (PTA 76-90 dB HL) | | | (PTA > 90 dB HL) | |
| | Stimulation level | | | | | | | | | | | | | | | | | | | |
| Frequency | S^{a}₀ [symbol] | S₀ | S₀ | S₄ | S₀ | S₀ | S₀ | S₄ | S₀ | S₁₀ | S₁₀ | S₁₀ | S₁₀ | S₁₀ | S₁₀ | S₁₀ | S₁₀ | S₁₀ | S₁₀ | S₁₀ |
| 1, 2 & [symbol] 4 kHz | 0 | 5 | 10 | 15 | 20 | 25 | 30 | 35 | 40 | 45 | 50 | 55 | 60 | 65 | 70 | 75 | 80 | 85 | 90 | 95 |
| 0.5 kHz | 5 | 10 | 15 | 20 | 25 | 30 | 35 | 40 | 45 | 50 | 55 | 60 | 65 | 70 | 75 | 80 | 85 | 90 | 95 | 100 |

Note:
PTA = pure-tone average,
dB HL = decibel hearing level
^{a}Based on the modified Siegel criteria
^{b}Stratified Hearing Scale [symbol]
indicates data missing or illegible when filed

iOS Automated Audiometry Calibration

To calibrate the sound output of the iOS mobile devices to a hearing threshold level of zero at various frequencies, we applied the reference equivalent threshold sound pressure levels (RETSPLs) for Apple EarPods, which were reported in a prior study with consistent output across different EarPod pairs and between the right and left earphones and therefore could be applied to various Apple mobile devices with EarPods [29]. To record the eardrum pressure and evaluate the sound quality, the EarPods were placed in the left and right pinna of a KEMAR manikin, which included a head and torso that had been designed specifically for anthropomorphic testing in the audiologic industry [30]. The microphones of the simulators and the electrical and acoustical measurement systems were calibrated using a GRAS model 42AA pistonphone. The hearing thresholds were determined in an ascending order, as described in ISO 8253-1 [31], with a step size of 1 dB. The initial stimulus level was set to be 10 dB lower than the lowest subject response threshold, which was predetermined by conventional audiometry. Pure-tone stimuli at 0.25 kHz, 0.5 kHz, 1 kHz, 2 kHz, 4 kHz, and 8 kHz were generated on the iOS mobile devices and delivered by the Apple EarPods. All the devices were standardized by setting the user-controllable volume to 100% of the maximum limit. A 2-down, 1-up adaptive staircase procedure was used to determine the final hearing threshold of each subject after 3 reversals [32]. The maximum output difference between the right and left EarPods was less than 1 dB HL, and the maximum output difference between the devices (iPhone 7 and iPhone 7 Plus) was less than 1.5 dB HL. The output levels of the EarPods were calibrated in units of dB sound pressure level (SPL) when the volume of the Apple mobile device was set to the maximum. The output level (dB HL) of the pure tone at each test frequency was similar to that previously reported [28,29].

Hearing Screening Procedures

FIG. 4 shows how the proposed Ear Scale app was used for hearing screening among patients enrolled in this study who had signs of possible sudden hearing loss. Participants underwent the Ear Scale app examination at presentation and were classified into three groups (≤S₅, S₆-S₁₀, >S₁₀) based on their test results. We then arranged comprehensive hearing assessments, including otoscopy, conventional pure-tone audiometry, and other examinations for those who had a hearing scale greater than S₁₀ in one ear or asymmetrical hearing with hearing scale differences greater than 5-scale between the affected ear and the contralateral ear (FIG. 4). Participants with bilateral sudden sensorineural hearing loss or conductive hearing loss were excluded from the study population.

Statistical Analysis

Pearson's correlation coefficients were estimated to investigate the correlation between PTA measured by conventional pure-tone audiometry and the Hearing Scale derived from the Ear Scale app using the HST, as well as the differences in the hearing results between the impaired ear and the contralateral ear of each individual. The corresponding PTA of each Hearing Scale group was demonstrated using a box plot. ANOVA was used to determine the difference in the mean PTA between each scale. Indicators of validity and the predictive value were estimated to determine the diagnostic accuracy of the HST for SSNHL compared to that of the gold standard pure-tone audiometric evaluation. Patients with a PTA difference between the two ears of at least 30 dB HL within a 72-hour period (i.e., the diagnostic gold standard for identifying SSNHL that was used in this study), as assessed by conventional pure-tone audiometry, were considered positive for SSNHL. We then estimated the sensitivity, specificity, positive predictive value (PPV), and negative predictive value (NPV) for diagnosing SSNHL on the basis of three different hearing scale differences (5-scale difference, 6-scale difference, and 7-scale difference) between the two ears, as measured by the Ear Scale app. Sensitivity was defined as the percentage of individuals with true SSNHL (i.e., patients with PTA thresholds that met the diagnostic gold standard for the presence of SSNHL included in the American Academy of Otolaryngology-Head and Neck Surgery guidelines [1]) who were correctly identified as having SSNHL by the Ear Scale app. PPV was defined as the probability of true SSNHL being present among participants who were considered positive for SSNHL by the Ear Scale app. The significance tests for all analyses were 2-sided and included a type I error of 0.05. The power was set to be 0.8. The statistical software used was Stata 15 (StataCorp, College Station, Tex.).

Results

Baseline Characteristics of the Study Sample

This study included 88 adults with possible SSNHL who visited the emergency department or an otolaryngology clinic from January 2018 to June 2019; patients with bilateral or conductive hearing loss were excluded. The mean age of the study cohort was 46 years, and 50% were females (Table 2). The average PTA of the cohort included in the analytic cohort was 67.1 dB HL (Table 2). The average hearing scale measured by the Ear Scale app was S₁₇ (i.e., 85 dB HL). Regarding the differences in the hearing results between the two ears, the mean PTA difference was 47.6 dB HL, whereas the average Hearing Scale difference (obtained from the Ear Scale app) was 9 hearing scales (i.e., 45 dB HL) (Table 2).

TABLE 2
Baseline characterisitics of the study sample

| Variables | Statistics |
|---|---|
| Total sample (n) | 88 |
| Age, years (mean ± SD) | 46 ± 14.7 |
| Gender n (%) | |
| Female | 44 (60) |
| Pretreatment hearing grade of worst-hearing, n (%) | |
| Grade 1 (PTA ≤ 25 dB HL) | 7 (6) |
| Grade 2 (PTA 26-45 dB HL) | 8 (9) |
| Grade 3 (PTA 46-75 dB HL) | 43 (40) |
| Grade 4 (PTA 76-90 dB HL) | 16 (16) |
| Grade 5 (PTA > 90 dB HL) | 14 (16) |
| PTA of worst-hearing ear, dB (mean ± SD) | 67.1 ± 24.9 |
| Average scale worst-hearing ear, dB (mean ± SD) | 17 ± 4 |
| Average PTA difference, dB (mean ± SD) | 47.6 ± 25.0 |
| Average scale difference (mean ± SD) | 9 ± 4 |

Note:
SD = standard deviation;
PTA = pure-tone average;
dB HL = declined hearing level
a. PTA difference = PTA of impaired ear − PTA of contralateral ear
b. Hearing Scale difference = Hearing Scale of impaired ear − Hearing Scale contralateral ear

Correlation Between the PTA and Hearing Scale

Pearson's correlation analyses revealed strong positive correlations between the PTA assessed by pure-tone audiometry and the Hearing Scale measured by the Ear Scale app as well as between the PTA differences and Hearing Scale differences between the two ears, with correlation coefficients of 0.88 (95% confidence interval [CI]=0.82-0.92) and 0.84 (95% CI=0.77-0.90), respectively (FIG. 2).

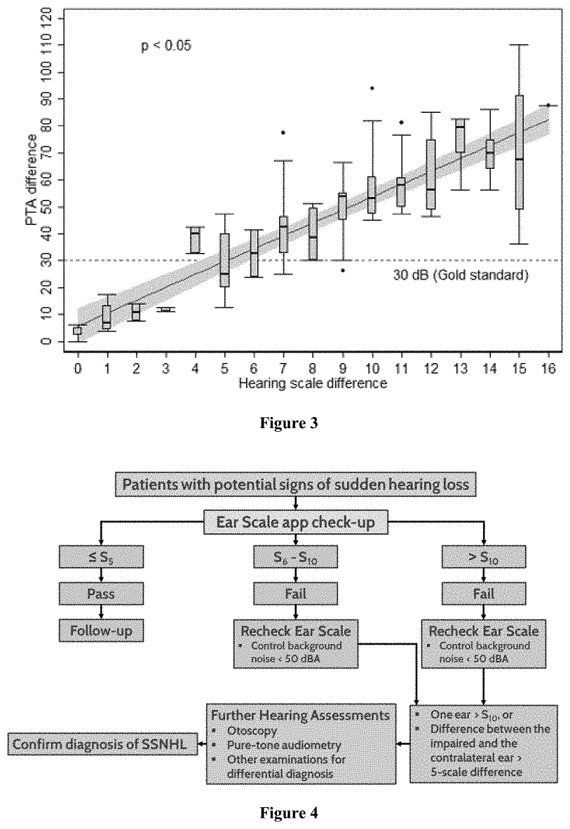

The association of the PTA and Hearing Scale differences between the two ears is presented in FIG. 3. The mean PTA difference differed significantly across the Hearing Scale groups (p<0.05).

Validity of the Ear Scale App in Diagnosing SSNHL

The diagnostic gold standard for SSNHL used in our study was a PTA difference between the impaired ear and the contralateral ear of greater than or equal to 30 dB HL. The indicators of validity of the Ear Scale app and the cut-off values for the hearing scale differences are displayed in Table 3. The 5-scale difference (i.e., 25 dB HL) had the highest sensitivity (95.5%, 95% CI=87.5%-99.1%) in diagnosing SSNHL, while the 7-scale difference (i.e., 35 dB HL) showed the highest specificity (90.5%, 95% CI=69.6%-98.8%).

TABLE 3
Diagnostic validity of the Hearing Scale difference^{a}

| | Sensitivity, % | Specificity, % | PPV, % | NPV, % |
|---|---|---|---|---|
| Hearing scale difference | (95% CI) | (95% CI) | (95% CI) | (85% CI) |
| 5 hearing scales (25 dB) | 95.5 (87.5-99.1) | 68.7 (43.0-85.4) | 99.1 (80.7-95.9) | 82.3 (56.6-96.2) |
| 6 hearing scales (30 dB) | 92.5 (83.4-97.5) | 85.7 (63.7-97.0) | 85.4 (87.1-99.0) | 78.3 (56.3-9.25) |
| 7 hearing scales (35 dB) | 91.0 (61.5-96.6) | 80.5 (69.6-98.8) | 98.6 (89.0-99.6) | 76.0 (54.9-90.8) |

Note:
dB HL = decibel hearing level;
CI = Confidence Interval;
PPV = Positive predictive value;
NPV = Negative predictive value
^{a}Hearing Scale difference = Hearing Scale of impaired ear − Hearing Scale contralateral ear

A previous study implemented the proposed Ear Scale app for hearing screening among a pediatric population and reported a strong correlation in the PTA between the app and conventional pure-tone audiometry in a soundproof booth as well as high accuracy in identifying school-aged children with hearing impairment [26]. Notably, Handzel et al. [33] utilized a different smartphone-based application, the uHear hearing test application, for the initial assessment of unilateral SSNHL in 32 patients who had been diagnosed with SSNHL by standard audiometry. Table 4 illustrates the comparison between the gold standard approach (i.e., pure-tone audiometry), the uHear app, and the proposed Ear Scale app in this study. The authors observed a sensitivity of 76% with the most stringent gold standard and of 94% with the least stringent criterion when they used the smartphone-based hearing screening tool for diagnosing SSNHL (Table 4) [33]. Our results were consistent with these findings, added to the literature by providing results in a larger sample size and better diagnostic validity, and further broadened the population eligible for hearing screening using the hearing scale difference between the impaired ear and the contralateral ear as measured by the Ear Scale app. A significant strength of the proposed method for evaluating SSNHL is that instead of measuring the exact hearing thresholds, we used the Hearing Scale difference between the two ears to identify SSNHL. A major concern in measuring hearing status using these smartphone- or tablet-based tools is the ambient noise level, since they are not administered in a soundproof booth like conventional pure-tone audiometry. The presence of background noise can negatively affect hearing performance and lead to inaccurate results. This problem was minimized with our approach, as the hearing scale difference between the two ears was used—the influence of ambient noise was therefore canceled out. This unique feature indicates that the implementation of the Ear Scale app can be feasible in noisy environments, thereby broadening its applicability to settings such as urgent care clinics or emergency departments.

TABLE 4
Comparison of key characteristics among different approaches for identifying SSNHL

| | | Audiometric | | | | Sensitivity/ |
|---|---|---|---|---|---|---|
| Diagnostic | | Criteria of | | Sample | Measurement | Specificity |
| Approach | Author | SSNHL | Role | Size | Unit | (%) |
| Conventional | Stachler | A decrease in | Gold | — | dB HL | — |
| pure-tone | et al. [1] | hearing of ≥30 [symbol] dB, | standard | | | |
| audiometry | | affecting at lease | | | | |
| | | 3 consecutive | | | | |
| | | frequencies^{a, b} | | | | |
| uHear hearing | Handzel | Hearing loss of at | Smartphone- | N = 32 | Hearing grade | 76.0/91.0 |
| test application | et al. | least 2 hearing | based test | | | |
| | [32] | grades across 3 | | | | |
| | | or more | | | | |
| | | consecutive | | | | |
| | | frequencies^{a, b} | | | | |
| Ear Scale | Lin et al. | Hearing loss of at | Smartphone- | N = 56 | Hearing scale | 95.5/66.7 |
| application | | least 5 hearing | based test | | | |
| (current study) | | scales difference^{b} | | | | |

Note:
SSNHL = sudden sensorineural hearing loss;
dB HL = decibel hearing level;
PTA = pure-tone average
^{a}The definition by the American Academy of Ototaryngology-Head and Neck Surgery guideline [1]
^{b}Hearing loss is defined as related to the opposite ear's thresholds
^{c}The hearing thresholds are grouped into 6 grades (American Speech-Language-Hearing Association 2812, normal = B-25 dB, mild = 26-40 dB HL, moderate = 41-55 dB HL, moderately severe = 56-70 dB HL, severe = 71-90 dB HL and profound > 90 dB HL) [symbol]
indicates data missing or illegible when filed

Several practice guidelines and reviews have suggested that patients with possible SSNHL undergo a comprehensive clinical workup upon arrival in the clinic, including thorough history taking, relevant physical examinations, and tuning fork tests to differentiate other types of hearing loss from SSNHL, identify non-idiopathic etiologies, and generate differential diagnosis [1,5,34,35]. Although these approaches are important and convenient, they can yield unreliable, even misleading, results [36,37]. Audiometric confirmation is still mandatory for definitively diagnosing SSNHL and should be performed on an emergent basis [1,5]. Conventional pure-tone audiometry remains the preferred method because it accurately distinguishes conductive hearing loss from those of sensorineural origins and establishes frequency-specific hearing thresholds, which are required components of frequently used audiometric criteria for SSNHL [1, 5]. Initial audiometric outcomes also provide information essential for predicting prognoses and planning treatments [1]. Given their critical role in the management of SSNHL, audiometric assessments should be performed in accordance with the protocols proposed by the American Speech-Language-Hearing Association and the standards regarding the maximum allowable ambient noise and proper calibration [1,38,39]. In primary care practices (PCPs) or other busy clinical settings, such as urgent care and emergency departments (EDs), performing a standard battery of audiology tests can be challenging [13, 40]. The high costs of equipment, limited space and time, noisy environments, and shortage of qualified personnel who are capable of conducting the screening and daily health assessments with audiometry are barriers to conventional pure-tone evaluations [13, 40, 41]. In a study that investigated the procedures performed by general practitioners working in primary care clinics, less than 20% of clinicians performed audiometry in their practices [42]. Since conventional pure-tone audiometry is mostly unavailable in PCP settings, innovative telehealth approaches have emerged that have been demonstrated as applicable and cost-effective for hearing assessments in PCP-level settings [14,41,43,44]. The Ear Scale app proposed in this study has been shown to be feasible for hearing screenings in the pediatric population [26] and has been shown to have a good level of diagnostic accuracy for SSNHL. It is suggested in the present invention that this is a novel tool, which incorporates the HST and smartphone-based technology, can serve as a point-of-care test for SSNHL at the PCP level because it is affordable, efficient, and requires minimal training to administer. The proposed procedure used in this study (FIG. 4) could be the standardized approach when implementing the Ear Scale app in real-world settings. It could therefore assist healthcare providers in PCP or urgent care settings in making appropriate decisions regarding otolaryngology referrals, reduce the possibility of delayed management, and potentially improve the hearing recovery of patients with SSNHL.

Since the premorbid hearing status is generally unknown among people with possible SSNHL, hearing loss is usually defined on the basis of the difference between the two ears in the thresholds [1]. Based on our results, the PTA differences and hearing scale differences between the two ears are strongly correlated, with a correlation coefficient of 0.84. The diagnostic validity of three selected hearing scale difference cut-offs is reported in our study. Although all three cut-off values yielded satisfactory sensitivity, we preferred and recommended using the 5-hearing scale difference, as it had the lowest false-negative responses and can serve as the diagnostic standard for SSNHL. There is evidence that patients with untreated/unrecovered SSNHL have more tinnitus and balance problems as well as a poorer long-term quality of life [6,45]. These findings pose significant concerns regarding other negative health consequences associated with hearing loss, including falls [9,46], social isolation [47], depression [48], and incident dementia [10]. In the presence of other common sources of hearing loss, such as presbycusis, the impact of SSNHL is aggravated. In addition, misclassifying diseased cases as nondiseased cases may lead to delayed care among individuals with SSNHL, which is an important prognostic factor because it can be prevented [7,8]. Given that further hearing evaluations of SSNHL, which mainly include standard pure-tone audiometric assessments, are neither invasive nor harmful, minimizing the false-negative rate should be the goal of adequate tools when screening persons with possible SSNHL.

It is found in the present invention, the hearing results measured by the smartphone-based Ear Scale app according to the present invention was strongly correlated to the results measured by the conventional pure-tone audiometry among patients with possible SSNHL. In addition, the hearing scale difference between the two ears, as measured by the Ear Scale app, had a satisfactory level of validity in detecting SSNHL. It is suggested that this smartphone-based approach may effectively assist the evaluation of SSNHL in clinical settings where conventional pure-tone audiometry is not available.

All publications, patents, and patent documents cited herein above are incorporated by reference herein, as though individually incorporated by reference.

The invention has been described with reference to various specific and preferred embodiments and techniques. However, one skilled in the art will understand that many variations and modifications may be made while remaining within the spirit and scope of the invention.

### REFERENCES

1. Stachler R J, Chandrasekhar S S, Archer S M, et al. Clinical practice guideline: Sudden hearing loss. Otolaryngology—head and neck surgery. 2012; 146(3 Suppl):S1-S35. https://www.ncbi.nlm.nih.gov/pubmed/22383545. doi: 10.1177/0194599812436449.
2. Alexander T, Harris J. Incidence of sudden sensorineural hearing loss. Otology & Neurotology. 2013; 34(9):1586-1589. http://ovidsp.ovid.com/ovidweb.cgi?T=JS&NEWS=n&CSC=Y&PAGE=fulltext&D=ovft&AN=00129492-201312000-00008. doi: 10.1097/MAO.0000000000000222.
3. Byl F M. Seventy-six cases of presumed sudden hearing loss occurring in 1973: Prognosis and incidence. The Laryngoscope. 1977; 87(5):817-825. https://onlinelibrary.wiley.com/doi/abs/10.1002/lary.5540870515. doi: 10.1002/lary.5540870515.
4. Kuo C, Chung C, Wang C, Chien W, Chen H. Increased incidence in hospitalised patients with sudden sensorineural hearing loss: A 14-year nationwide population-based study. International Journal of Audiology. 2019; 58(11):769-773. http://www.tandfonline.com/doi/abs/10.1080/14992027.2019.1627009. doi: 10.1080/14992027.2019.1627009.
5. Kuhn M, Heman-Ackah S E, Shaikh J A, Roehm P C. Sudden sensorineural hearing loss. Trends in Amplification. 2011; 15(3):91-105. https://journals.sagepub.com/doi/full/10.1177/1084713811408349. doi: 10.1177/1084713811408349.
6. Härkönen K, Kivekäs I, Rautiainen M, Kotti V, Vasama J. Quality of life and hearing eight years after sudden sensorineural hearing loss. The Laryngoscope. 2017; 127(4):927-931. https://onlinelibrary.wiley.com/doi/abs/10.1002/lary.26133. doi: 10.1002/lary.26133.
7. Edizer D T, Çelebi Ö, Hamit B, Baki A, Yi\check{g}it Ö. Recovery of idiopathic sudden sensorineural hearing loss. The journal of international advanced otology. 2015; 11(2):122-126. https://www.ncbi.nlm.nih.gov/pubmed/26381001. doi: 10.5152/iao.2015.1227.
8. Cvorovi\ddot{c} L, Deric D, Probst R, Hegemann S. Prognostic model for predicting hearing recovery in idiopathic sudden sensorineural hearing loss. Otology & neurotology. 2008; 29(4):464-469. https://www.ncbi.nlm.nih.gov/pubmed/18434930. doi: 10.1097/MAO.0b013e31816fdcb4.
9. Deal J A, Richey Sharrett A, Bandeen-Roche K, et al. Hearing impairment and physical function and falls in the atherosclerosis risk in communities hearing pilot study. Journal of the American Geriatrics Society. 2016; 64(4):906-908. https://onlinelibrary.wiley.com/doi/abs/10.1111/jgs.14075. doi: 10.1111/jgs.14075.
10. Lin F R, Metter E J, O'Brien R J, Resnick S M, Zonderman A B, Ferrucci L. Hearing loss and incident dementia. Archives of neurology (Chicago). 2011; 68(2):214-220. http://dx.doi.org/10.1001/archneurol.2010.362. doi: 10.1001/archneurol.2010.362.
11. Deal J A, Sharrett A R, Albert M S, et al. Hearing impairment and cognitive decline: A pilot study conducted within the atherosclerosis risk in communities neurocognitive study. American journal of epidemiology. 2015; 181(9):680-690. https://www.ncbi.nlm.nih.gov/pubmed/25841870. doi: 10.1093/aje/kwu333.
12. Reed N S, Altan A, Deal J A, et al. Trends in health care costs and utilization associated with untreated hearing loss over 10 years. JAMA otolaryngology—head & neck surgery. 2019; 145(1):27-34. http://dx.doi.org/10.1001/jamaoto.2018.2875. doi: 10.1001/jamaoto.2018.2875.
13. Louw C, Swanepoel D W, Eikelboom R H. Self-reported hearing loss and pure tone audiometry for screening in primary health care clinics. Journal of primary care & community health. 2018; 9:215013271880315-2150132718803156. https://search.datacite.org/works/10.1177/2150132718803156. doi: 10.1177/2150132718803156.
14. Clark J L, Swanepoel D W. Technology for hearing loss—as we know it, and as we dream it. Disability and Rehabilitation: Assistive Technology. 2014; 9(5):408-413. http://www.tandfonline.com/doi/abs/10.3109/17483107.2014.905642. doi: 10.3109/17483107.2014.905642.
15. Ratanjee-Vanmali H, Swanepoel D W, Laplante-Lévesque A. Patient uptake, experience, and satisfaction using web-based and face-to-face hearing health services: Process evaluation study. Journal of medical Internet research. 2020; 22(3):e15875. https://www.ncbi.nlm.nih.gov/pubmed/32196459. doi: 10.2196/15875.
16. Masalski M, Krecicki T. Self-test web-based pure-tone audiometry: Validity evaluation and measurement error analysis. Journal of medical Internet research. 2013; 15(4):e71. https://www.ncbi.nlm.nih.gov/pubmed/23583917. doi: 10.2196/jmir.2222.
17. Liao W, Young S, Lien C, Wang S. An audiometer to monitor progressive hearing change in school-aged children. Journal of Medical Screening. 2011; 18(1):8-11. https://journals.sagepub.com/doi/full/10.1258/jms.2011.010120. doi: 10.1258/jms.2011.010120.
18. Honeth L, Bexelius C, Eriksson M, et al. An internet-based hearing test for simple audiometry in nonclinical settings: Preliminary validation and proof of principle. Otology & neurotology. 2010; 31(5):708-714. https://www.ncbi.nlm.nih.gov/pubmed/20458255. doi: 10.1097/MAO.0b013e3181de467a.
19. Yimtae K, Israsena P, Thanawirattananit P, et al. A tablet-based mobile hearing screening system for preschoolers: Design and validation study. JMIR mHealth and uHealth. 2018; 6(10):e186. https://search.datacite.org/works/10.2196/mhealth.9560. doi: 10.2196/mhealth.9560.
20. Masalski M, Grysiński T, Krȩcicki T. Hearing tests based on biologically calibrated mobile devices: Comparison with pure-tone audiometry. JMIR mHealth and uHealth. 2018; 6(1):e10. https://www.ncbi.nlm.nih.gov/pubmed/29321124. doi: 10.2196/mhealth.7800.
21. Sandström J, Swanepoel D W, Carel Myburgh H, Laurent C. Smartphone threshold audiometry in underserved primary health-care contexts. International Journal of Audiology. 2016; 55(4):232-238. http://www.tandfonline.com/doi/abs/10.3109/14992027.2015.1124294. doi: 10.3109/14992027.2015.1124294.
22. Masalski M, Kipiński L, Grysiński T, Krȩcicki T. Hearing tests on mobile devices: Evaluation of the reference sound level by means of biological calibration. Journal of medical Internet research. 2016; 18(5):e130. https://www.ncbi.nlm.nih.gov/pubmed/27241793. doi: 10.2196/jmir.4987.
23. Bright T, Pallawela D. Validated smartphone-based apps for ear and hearing assessments: A review. JMIR rehabilitation and assistive technologies. 2016; 3(2):e13. https://www.ncbi.nlm.nih.gov/pubmed/28582261. doi: 10.2196/rehab.6074.
24. Liao W, Lien C, Young S. The hearing scale test for hearing screening of school-age children. International Journal of Pediatric Otorhinolaryngology. 2010; 74(7):760-764. https://www.clinicalkey.es/playcontent/1-s2.0-S016558761000128X. doi: 10.1016/j.ijpor1.2010.03.033.
25. Wen-Huei Liao, Shuenn-Tsong Young, Shih-Tsang Tang, et al. A novel method for quick hearing assessment of children. ICEIE. August 2010; 1:V1-37.
26. Chu Y, Cheng Y, Lai Y, et al. A mobile phone-based approach for hearing screening of school-age children: Cross-sectional validation study. JMIR mHealth and uHealth. 2019; 7(4):e12033. https://www.ncbi.nlm.nih.gov/pubmed/30932870. doi: 10.2196/12033.
27. Cheng Y, Chu Y, Tu T, Shiao A, Wu S, Liao W. Modified siegel's criteria for sudden sensorineural hearing loss: Reporting recovery outcomes with matched pretreatment hearing grades. Journal of the Chinese Medical Association. 2018; 81(11):1008-1012. http://dx.doi.org/10.1016/j.jcma.2018.03.012. doi: 10.1016/j.jcma.2018.03.012.
28. Foulad A, Bui P, Djalilian H. Automated audiometry using apple iOS-based application technology. Otolaryngology—head and neck surgery. 2013; 149(5):700-706. https://search.datacite.org/works/10.1177/0194599813501461. doi: 10.1177/0194599813501461.
29. Ho C, Li P, Young S. Reference equivalent threshold sound pressure levels for apple EarPods. The Journal of the Acoustical Society of America. 2017; 141(2):EL115-EL119. https://search.datacite.org/works/10.1121/1.4976110. doi: 10.1121/1.4976110.
30. Gardner W G, Martin K D. HRTF measurements of a KEMAR. J Acoust Soc Am 1995 June; 97(6):3907-3908.
31. International Organization for Standardization. Reference equivalent threshold sound pressure levels for Apple EarPods URL:https://www.iso.org/standard/43601.html [accessed 2020-07-21] [WebCite Cache ID 76YADA31k]
32. Levitt H. Transformed Up-Down methods in psychoacoustics. The Journal of the Acoustical Society of America. 1971; 49(2B):467-477. https://search.datacite.org/works/10.1121/1.1912375. doi: 10.1121/1.1912375.
33. Handzel O, Ben-Ari O, Damian D, Priel M M, Cohen J, Himmelfarb M. Smartphone-based hearing test as an aid in the initial evaluation of unilateral sudden sensorineural hearing loss. Audiology & neurotology. 2013; 18(4):201-207. https://search.datacite.org/works/10.1159/000349913. doi: 10.1159/000349913.
34. Jensen E A, Harmon E, Smith W. Early identification of idiopathic sudden sensorineural hearing loss. The Nurse Practitioner. 2017; 42(9):10-16. https://www.ncbi.nlm.nih.gov/pubmed/28787350. doi: 10.1097/01.NPR.0000521990.15440.ee.
35. Burgess L P, Frankel S F, Lepore M L, Yim D W. Tuning fork screening for sudden hearing loss. Military medicine. 1988; 153(9):456-458. https://www.ncbi.nlm.nih.gov/pubmed/3141836. doi: 10.1093/milmed/153.9.456.
36. Stankiewicz J A, Mowry H J. Clinical accuracy of tuning fork tests. The Laryngoscope. 1979; 89(12):1956-1963. https://onlinelibrary.wiley.com/doi/abs/10.1288/00005537-197912000-00009. doi: 10.1288/00005537-197912000-00009.
37. Browning G G, Swan I R C, Chew K K. Clinical role of informal tests of hearing. The Journal of Laryngology & Otology. 2007; 103(1):7-11. https://search.datacite.org/works/10.1017/s0022215100107923. doi: 10.1017/s0022215100107923.
38. Preferred practice patterns for the professions of speech-language pathology and audiology. american speech-language-hearing association. ASHA. Supplement. 1993; 35(3 Suppl 11):i. https://www.ncbi.nlm.nih.gov/pubmed/8097652.
39. Frank T. ANSI update: Maximum permissible ambient noise levels for audiometric test rooms. American Journal of Audiology. 2000; 9(1):3-8. https://pubs.asha.org/doi/10.1044/1059-0889(2000/003). doi: 10.1044/1059-0889(2000/003).
40. Bettger J P, Dolor R J, Witsell D L, et al. Comparative implementation-effectiveness of three strategies to perform hearing screening among older adults in primary care clinics: Study design and protocol. BMC geriatrics. 2020; 20(1):170. https://www.ncbi.nlm.nih.gov/pubmed/32393184. doi: 10.1186/s12877-020-01576-x.
41. Louw C, Swanepoel D W, Eikelboom R H, Myburgh H C. Smartphone-based hearing screening at primary health care clinics. Ear and hearing. 2017; 38(2):e93-e100. https://search.datacite.org/works/10.1097/aud.0000000000000378. doi: 10.1097/aud.0000000000000378.
42. Flaegel K, Brandt B, Goetz K, Steinhaeuser J. Which procedures are performed by general internists practicing primary care in germany?—a cross-sectional study. BMC family practice Web site: https://search.datacite.org/works/10.6084/m9.figshare.c.4958546. Updated 2020.
43. Swanepoel D W, Myburgh H C, Howe D M, Mahomed F, Eikelboom R H. Smartphone hearing screening with integrated quality control and data management. International Journal of Audiology. 2014; 53(12):841-849. https://search.datacite.org/works/10.3109/14992027.2014.920965. doi: 10.3109/14992027.2014.920965.
44. Yousuf Hussein S, Wet Swanepoel D, Biagio de Jager L, Myburgh H C, Eikelboom R H, Hugo J. Smartphone hearing screening in mHealth assisted community-based primary care. Journal of telemedicine and telecare. 2016; 22(7):405-412. https://search.datacite.org/works/10.1177/1357633x15610721. doi: 10.1177/1357633x15610721.
45. Chiossoine-Kerdel J A, Baguley D M, Stoddart R L, Moffat D A. An investigation of the audiologic handicap associated with unilateral sudden sensorineural hearing loss. The American journal of otology (New York, N.Y.). 2000; 21(5):645. https://www.ncbi.nlm.nih.gov/pubmed/10993452.
46. Lin F R, Ferrucci L. Hearing loss and falls among older adults in the United States. Archives of internal medicine (1960). 2012; 172(4):369-371. http://dx.doi.org/10.1001/archinternmed.2011.728. doi: 10.1001/archinternmed.2011.728.
47. Shukla A, Harper M, Pedersen E, et al. Hearing loss, loneliness, and social isolation: A systematic review. Otolaryngology-Head and Neck Surgery. 2020; 162(5):622-633. https://journals.sagepub.com/doi/full/10.1177/0194599820910377. doi: 10.1177/0194599820910377.
48. Shukla A, Reed N, Armstrong N M, Lin F R, Deal J A, Goman A M. Hearing loss, hearing aid use and depressive symptoms in older adults—findings from the atherosclerosis risk in communities neurocognitive study (ARIC-NCS). The journals of gerontology. Series B, Psychological sciences and social sciences. 2019. https://search.datacite.org/works/10.1093/geronb/gbz128. doi: 10.1093/geronb/gbz128.

What is claimed is:

1. a computer-implanted method for diagnosing sudden sensorineural hearing loss (SSNHL) of an ear in a subject; comprising on one or more processors of the computer the steps of:
(a) measuring environmental background noise;
(b) providing a test tone at a high frequency using a plurality of magnitude levels for testing the hearing of a first ear of the subject, wherein the test tone is a pure tone;
(c) if the subject passes the hearing test in the step (b), providing a plurality of test tones using a plurality of magnitude levels respectively for testing the hearing of the first ear of the subject to record the results as a first hearing scale;
(d) providing a plurality of test tones using a plurality of magnitude levels for testing the hearing of a second ear of the subject to record the results as a second hearing scale, wherein the test tone is a pure tone;
(e) calculating the difference between the first hearing scale and the second hearing scale; and
(f) comparing the difference with a threshold to diagnose the subject as SSNHL or not; wherein the threshold is defined as the difference being five magnitude levels.

2. The method of claim 1, wherein the computer is a smartphone.

3. The method of claim 1, wherein the test tone is 8000 Hz, 4000 Hz, 2000 Hz, 1000 Hz, or 500 Hz.

4. The method of claim 1, wherein the difference of two magnitude levels is 5 decibel hearing level (dB HL).

5. The method of claim 1, wherein the threshold is the difference between the first hearing scale and the second hearing scale not less than 5 magnitude levels.

6. The method of claim 5, wherein the threshold is 25 dB HL.

7. A computer aid system configured to diagnose SSNHL, the computer aid system comprising:
a memory;
a microphone;
a headphone; and
one or more processors configured to perform the steps of:
(a) measuring environmental background noise;
(b) providing a test tone at a high frequency using a plurality of magnitude levels for testing the hearing of a first ear of the subject, wherein the test tone is a pure tone;
(c) if the subject passes the hearing test in the step (b), providing a plurality of test tones using a plurality of magnitude levels respectively for testing the hearing of the first ear of the subject to record the results as a first hearing scale;
(d) providing a plurality of test tones using a plurality of magnitude levels for testing the hearing of a second ear of the subject to record the results as a second hearing scale, wherein the test tone is a pure tone;
(e) calculating the difference between the first hearing scale and the second hearing scale; and
(f) comparing the difference with a threshold to diagnose the subject as SSNHL or not; wherein the threshold is defined as the difference being five magnitude levels.

8. The system of claim 7, wherein the computer is a desk computer, a notebook computer, a smartphone or a pad.

9. The system of claim 7, wherein the one or more processors are implanted in a smartphone.

10. The system of claim 7, wherein the test tone is a pure tone at a frequency at 8000 Hz, 4000 Hz, 2000 Hz, 1000 Hz or 500 Hz.

11. The system of claim 7, wherein the difference of two magnitude levels is 5 decibel hearing level (dB HL).

12. The system of claim 7, wherein the threshold is the difference between the first hearing scale and the second hearing scale not less than 5 magnitude levels.

13. The system of claim 12, wherein the threshold is 25 dB HL.
